(19) United States
(12) Patent Application Publication
(10) Pub. No.: US 2023/0233446 A1
(43) Pub. Date: Jul. 27, 2023

(54) FACIAL COSMETIC COMPOSITIONS AND METHODS OF IMPROVING FACIAL AESTHETICS

(71) Applicant: Aforé LLC, Libertyville, IL (US)

(72) Inventors: Julius Few, Chicago, IL (US); Alec Semersky, Libertyville, IL (US)

(21) Appl. No.: 18/160,072

(22) Filed: Jan. 26, 2023

Related U.S. Application Data
(60) Provisional application No. 63/303,837, filed on Jan. 27, 2022.

(51) Int. Cl. A61K 8/9789; A61K 8/67; A61K 8/34; A61K 8/64; A61K 8/73; A61Q 19/08

(52) U.S. Cl. CPC A61K 8/9789; A61K 8/671; A61K 8/347; A61K 8/64; A61K 8/735; A61Q 19/08

(57) ABSTRACT

Facial cosmetic compositions include a cannabinoid and a retinoid. The compositions can also comprise an aliphatic oligopeptide, an acetylated oligopeptide, and/or hyaluronic acid. Methods of administering facial cosmetic compositions include topically and/or subtopically administering to facial skin of a subject.

### CROSS-REFERENCE TO RELATED APPLICATIONS

This application claims the benefit under 35 U.S.C. § 119(e) of U.S. Provisional Patent Application Ser. No. 63/303,837, filed Jan. 27, 2022, the content of which is incorporated by reference herein in its entirety

### BACKGROUND

Skin aging is influenced by a number of factors, including extrinsic factors like exposure to UV light, poor diet, or pollutants, and intrinsic factors such as collagen and elastin loss over time. Zhang et al. (2018). Within the skin, intrinsic aging is due, in part, to the decreased capacity of keratinocytes, fibroblasts, and melanocytes to proliferate and the degeneration of the fibrous extracellular matrix, while 80% of extrinsic aging can be attributed to UV radiation. Dimri et al. (1995); Kohl et al. (2011); Friedman (2005). Interestingly, for both intrinsic and extrinsic aging, reactive oxygen species (ROS) and associated oxidative stress can accelerate these processes in the skin through multiple mechanisms and contribute to dyspigmentation, reduced barrier function, laxity, and uneven texture. Zhang et al. (2018); Rinnerthaler et al. (2015). In addition, ROS are associated with inflammation, which can further negatively impact the skin's appearance. Rinnerthaler et al. (2015); Tsuchida et al. (2020)

In clinical practice, one of the few topical treatments for which there is strong clinical evidence of efficacy for reducing the signs of aging is retinol.⁷ Mukherjee et al. (2006). The anti-aging effects of retinol are manifested by promoting proliferation of keratinocytes, strengthening the epidermis, and increasing collagen; however, retinol may indirectly decrease the antioxidative effects of the skin, leaving it prone to damage from ROS. Zasada et al. (2019); Hubbard et al. (2014); Wang et al. (2013). Furthermore, skin reactions such as redness, drying, peeling, or burning associated with retinol treatment can lead to inconsistent use or discontinuation by patients.

There remains an unmet need for a facial cosmetic composition that is efficacious in reducing or eliminating signs of aging without causing severe adverse events, in most if not all subjects.

### SUMMARY

As one aspect of the present invention, facial cosmetic compositions are provided. The compositions comprise (a) a cannabinoid and (b) a retinoid. The compositions can also comprise one or more (or all) of (c) an aliphatic oligopeptide; (d) an acetylated oligopeptide; and (e) hyaluronic acid. For example, the cannabinoid can be cannabidiol (CBD), a precursor or derivative of cannabidiol (such as Cannabidiolic Acid (CBDA)), or a mixture thereof. The retinoid can be retinol, retinal, a retinyl ester, or a mixture thereof. The aliphatic oligopeptide can be a palmitoyl oligopeptide such as palmitoyl pentapeptide-4, and the acetylated oligopeptide can be an acetyl hexapeptide such as acetyl hexapeptide-3. In some embodiments, components (a) through (e) are present in the particular amounts or concentrations disclosed herein. The present compositions can be formulated as creams, lotions, serums, oils, salves, liniments, ointments, gels, pastes, or facial masks. In some embodiments, the composition has a selected viscosity, as discussed herein.

As another aspect of the present invention, methods are provided for improving one or more facial aesthetics of a subject. The methods comprise applying to facial skin of a subject one of the facial cosmetic compositions described herein, wherein the composition is applied at least once per day for at least 21 days, alternatively for at least 42 days. In some embodiments, the composition is a cream applied to the facial skin without removal.

As yet another aspect of the present invention, methods are provided for improving one or more facial aesthetics of a subject. The methods comprise applying a facial cosmetic composition to facial skin of a subject, wherein the composition comprises (a) a cannabinoid and (b) a retinoid. The composition can also comprise one or more (or all) of (c) an aliphatic oligopeptide; (d) an acetylated oligopeptide; and (e) hyaluronic acid. The methods also comprise treating the facial skin of the subject by a dermatological procedure before, during or after applying the facial cosmetic composition. In some embodiments, the dermatological procedure is selected from the group consisting of microdermabrasion, hydradermabrasion, dermabrasion, and dermal planing. For example, the dermatological procedure can include abrading a surface of facial skin of a subject to remove dead cells and/or cell debris from the skin surface, and this abrading of the facial skin is performed before or during the application of the facial cosmetic composition to the facial skin. The abrading can be performed by techniques such as microdermabrasion and hydradermabrasion. In some embodiments, the facial cosmetic composition is applied to the facial skin by infusion, or by injection or microinjection.

These and other features and advantages of the present methods and compositions will be apparent from the following detailed description, in conjunction with the appended claims.

### BRIEF DESCRIPTION OF THE DRAWINGS

FIG. 1 shows components of the Aesthetic Global Ranking Scale used for evaluating the present facial cosmetic composition in Example 2.

FIG. 2 is a line graph that illustrates average differences observed in Example 2 for each of the 13 AGRS domains.

FIG. 3 shows results from Example 2 as average differences with confidence intervals around the paired differences.

FIG. 4 illustrates satisfaction and functional outcomes assessed in Example 2 using subject questionnaires completed on days 21 and 42.

FIGS. 5A and 5B illustrate how retinol and CBD from the present compositions can work synergistically to combat different causes of aging and improve facial aesthetics.

The present teachings are best understood from the following detailed description when read with the accompanying figures. The features are not necessarily drawn to scale. Wherever practical, like reference numerals refer to like features.

### DETAILED DESCRIPTION

The present compositions comprise a cannabinoid such as CBD and a retinoid such as retinol which work synergistically to combat different causes of aging. As explained below, retinoids such as retinol can provide anti-aging effects when applied to skin, but may also indirectly decrease the skin's endogenous protection against oxidative effects. By combining a retinoid with a cannabinoid such as CBD, antioxidative and anti-inflammatory effects can be produced, and the present compositions can act in a positive manner upon intrinsic and extrinsic factors involved in facial skin aesthetics.

It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

### Facial Cosmetic Compositions

The facial cosmetic compositions comprise (a) a cannabinoid and (b) a retinoid. The compositions can also comprise one or more (or all) of (c) an aliphatic oligopeptide; (d) an acetylated oligopeptide; and (e) hyaluronic acid. The term “cosmetic” refers to a material or action intended to restore or improve a person's appearance, such as preventing or reducing the signs of aging skin. Signs of aging skin include, in some instances, wrinkles, fine lines, blemishes, skin discoloration, dry skin, skin irritation, saggy skin, inelastic skin, enlarged pores, acne scars, inflammation, crow's feet, laugh lines, drooping eyelids, crepey skin, frown lines, dull skin tone, dark circles under the eyes, lackluster skin, itchy skin, hyperpigmentation, uneven skin tone, and collagen loss.

The term “cannabinoid” generally refers to chemical compounds that act on cannabinoid receptors of cells, thereby repressing neurotransmitter release in the brain and having other actions as well, such as in other tissues. Cannabinoids include the endocannabinoids (produced naturally in the body by humans and animals), the phytocannabinoids (found in Cannabis and some other plants), and synthetic cannabinoids (manufactured chemically). Cannabidiol (CBD) is a major constituent of the Cannabis plants, but there are over 80 different cannabinoids isolated from Cannabis, exhibiting varied effects. Cannabinoids are described and illustrated in LaRosa et al. US Pat. App. Publication No. 2021/0093539 A1 (especially at pages 2-5), the contents of which are incorporated by reference herein.

The cannabinoid included in the present compositions can be synthetically prepared, or alternatively, can be obtained naturally such as by extraction from plants. In some embodiments, the cannabinoid included in the present compositions has a desired purity (e.g., at least 95 wt. % pure, at least 98 wt. % pure, at least 99 wt. % pure, or at least 99.5 wt. % pure).

The terms “cannabidiol” and its abbreviation “CBD” refer to the compound cannabidiol having the chemical structure shown below, which is an exemplary cannabinoid for the present compositions, though natural or synthetic precursors and derivatives of cannabidiol are also contemplated such as cannabidiolic acid (CBDA) and other precursors or derivatives of cannabidiol. CBD is generally representative of cannabinoids to be employed in the present methods and compositions, and in the present disclosure, when an amount of CBD in a composition is recited, it should be understood that the amount can be adjusted if some or all of the CBD is present as CBDA or another precursor or derivative.

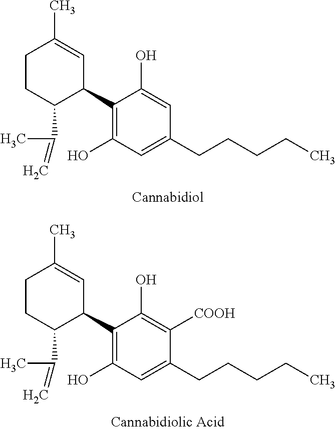

Other precursors and derivatives of cannabidiol (referred to as cannabidiol analogs) are described in Kennedy US Pat. App. Publication No. 20190216695 A1 and U.S. Pat. No. 6,630,507, the contents of which are incorporated by reference herein. Unless otherwise indicated, CBD refers to synthetic CBD as well as to CBD extracted from a Cannabis plant.

Another example of a cannabinoid is Cannabigerol (CBG) and its precursor Cannabigerolic acid (CBGA).

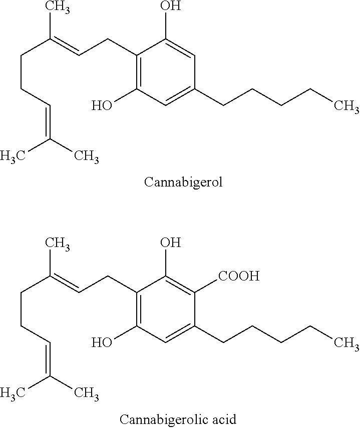 [SMILES: CCCCCc1cc(O)c(C/C=C(\C)CCC=C(C)C)c(O)c1 ; CCCCCc1cc(O)c(C/C=C(\C)CCC=C(C)C)c(O)c1C(=O)O]

In some embodiments, the present compositions comprise a cannabinoid selected from the group consisting of CBG, precursors and derivatives of CBG, and mixtures thereof. In some embodiments, the present compositions comprise a cannabinoid selected from the group consisting of CBD, CBG, precursors and derivatives of CBD and CBG, and mixtures thereof.

The present compositions can comprise one or more cannabinoids at a concentration (on a w/w basis) of at least 0.01%, or at least 0.1%, or at least 0.5%, or at least 0.75%, or at least 1%, or at least 1.1%, or at least 1.5%, or at least 2%, or at least 3%, or at least 5%, or at least 10%. The present compositions can comprise one or more cannabinoids at a concentration (on a w/w basis) of at most 20%, or at most 15%, or at most 10%, or at most 5%, or at most 4%, or at most 3%, or at most 2.5%, or at most 2%. Any of the foregoing minimum and maximum may be combined to form a desired concentration range, so long as the minimum is smaller than the maximum. When the cannabinoid is provided in a mixture (such as in a mixture comprising an oil or emulsifier), the weight of the cannabinoid itself (not including the oil or emulsifier or other components in the mixture) is used to calculate its percentage in the composition.

In some embodiments, the amount of cannabidiol (CBD) or other selected cannabinoid included in the present compositions is substantially pure cannabidiol, for example having less than 10% of unselected cannabinoids, or less than 5%, or less than 1%. The cannabinoid delta-9-tetrahydrocannabinol (THC) is the primary psychoactive compound of Cannabis. In some embodiments, the present compositions are substantially free of psychoactive cannabinoids, such as being substantially free of THC.

In some embodiments, the cannabinoid is added to a composition in the form of a cannabinoid nanoemulsion, microemulsion, or suspension. The cannabinoid can be provided as a salt, especially those cannabinoids which are carboxylic acids such as CBDA and CBGA. Examples of cannabinoid nanoemulsions include those disclosed in Gerardi et al. US Pat. App. Publication 20210177739; Spall et al. US Pat. App. Publication 20200245666; and Detzel US Pat. App. Publication 20200315965.

In some embodiments, the present compositions comprise a CBD extract as the cannabinoid. CBD extracts and methods of preparing CBD extracts are described in Mullen et al. US Pat. App. Publication No. 20210169759 A1, Garti et al. US Pat. App. Publication No. 20190231833 A1, and Marshall et al. US Pat. App. Publication No. 20190160393 A1, which are incorporated by reference herein.

The human endocannabinoid system (ECS) is composed of signaling molecules called endocannabinoids, receptors that bind them, and enzymes the modulate them. The endocannabinoid system plays a critical role in maintaining skin homeostasis and barrier function, and its dysregulation has been implicated in skin disorders (e.g., atopic dermatitis, psoriasis, acne, hyper/hypopigmentation). (Baswan 2020). Components of the ECS system are expressed in many skin cells, including epidermal keratinocytes, melanocytes, mast cells, fibroblasts, sebocytes, sweat gland cells, and hair follicles. (Toth 2019).

CBD has been said to modulate the ECS system and result in anti-aging effects. The phytocannabinoid CBD can modulate ECS activity by increasing levels of AEA (an endocannabinoid), which can affect cannabinoids signalling, including their interaction on cannabinoid receptors. (Atalay 2020). Depending on the concentration, CBD can activate, antagonize, or inhibit cannabinoid receptors (CB1 and CB2), ionotropic (TRP), and nuclear (PPAR) receptors. (Atalay 2020). Through modulation of the ECS system, CBD's anti-aging effects likely occur through its antioxidant and anti-inflammatory activity, as well as its effect of optimal sebum production (Casares 2020; Olah 2014; Petrosino 2017).

CBD induces expression of NRF2 target antioxidative genes, including robust expression of HMOX1 (Casares et al. 2020). CBD-induced HMOX1 expression was demonstrated in keratinocytes and in epidermis following topical application (Casares et al. 2020). HMOX1 expression is regulated by oxidative stress, which displaces transcriptional repressor BACH1, and allows nuclear NRF2 binding to the ARE motifs and subsequent transactivation of the HMOX1 gene (Saha 2020). HMOX1 catalyzes the rate-limiting step of oxidative heme degradation and plays crucial roles in modulating inflammation, apoptosis, and oxidative stress (Saha 2020; Casares 2020). Although CBD is a weak activator of NRF2, enhanced expression of HMOX1 by CBD does not occur directly through stabilization of NRF2 nor indirectly through ROS induction or through initiating an inflammatory response (Casares et al. 2020). Instead, CBD-induced activation of HMOX1 expression occurs through increased BACH1 degradation in a NRF2-independent mechanism that involves BACH1 nuclear export and cytosolic degradation (Casares et al. 2020).

The present compositions include one or more retinoids. Retinoids have been used for treating aging, burns, scaling, and dermatitis and are believed to regulate epithelial cell growth. Examples of retinoids include retinol, retinal (also called retinaldehyde), retinyl acetate, retinyl esters of fatty acids (such as retinyl palmitate, retinyl oleate, retinyl stearate, and retinyl linoleate), tretinoin, alitretinoin, isotretinoin, bexarotene, adapalene, and tazarotene. In some embodiments, the retinoid is selected from the group consisting of retinol (also called vitamin A1), retinal (also called vitamin A-aldehyde), retinyl esters (also called vitamin A-ester), and mixtures thereof.

Retinoids such as Vitamin A and its derivatives are among the most effective substances slowing the aging process. Through activation of retinoid nuclear receptors, retinoids regulate cellular apoptosis, differentiation and proliferation. Anti-wrinkle properties of retinoids promote keratinocytes proliferation, strengthen the protective function of the epidermis, restrain transepidermal water loss, protect collagen against degradation, promote collagen biosynthesis, and inhibit metalloproteinases activity (Zasada 2019; Hubbard 2014). Increased collagen content may then indirectly stimulate normalization of the elastic tissue organization (Hubbard 2014).

Retinol is particularly effective as it is fat soluble and can penetrate the stratum corneum and slightly into the dermis. When retinol enters into a keratinocyte, it binds CRBP or is transformed into more active metabolites, such as retinoic acid, which binds to CRABP2, enters into the nucleus, and activates its retinoid nuclear receptor. (Zasada 2019; Bouriez) The retinoid nuclear receptors, RAR and RXR, form heterodimers and function as transcriptional regulators. Activated RAR/RXR heterodimers can also interact with other transcription factors, such as AP-1, estrogen receptor α, and NF-kB (Wang 2007).

At low concentrations, retinoic acid prevents binding of NRF2 to the ARE, thereby suppressing the expression of NRF2-target antioxidative genes (Wang 2007). Furthermore, RXR can inhibit the transcriptional activity of NRF2 through associating with ARE-bound NRF2, suggesting that inhibition of NRF2 by RXR arises from direct interference of recruitment of coactivators to gene promoters (Wang 2013).

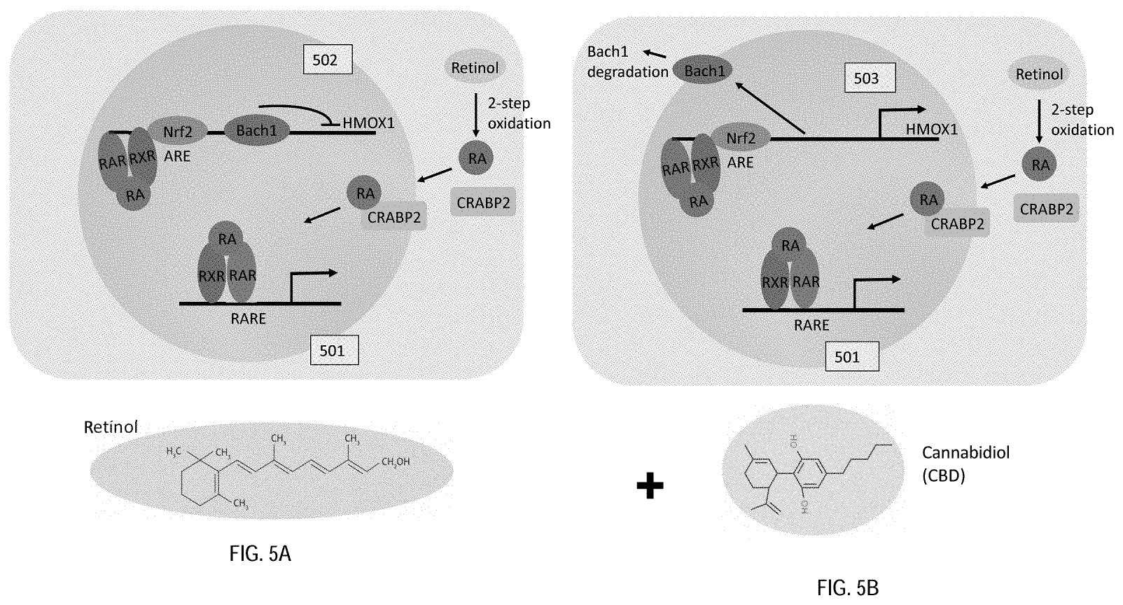

FIGS. 5A and 5B illustrate how retinol and CBD from the present compositions can work synergistically to combat different causes of aging and improve facial aesthetics. FIG. 51A illustrates how retinol's anti-aging effects are manifested through promoting keratinocytes proliferation, strengthening of the epidermis, and increasing collagen in the skin. 501: Transcription of genes involved keratinocytes proliferation, epidermis thickening, collagen biosynthesis/protection. However, retinol may indirectly decrease the skin's antioxidative effects through inhibition of NRF2 by activating the retinol nuclear factors. 502: No transcription of Nrf2 target antioxidative genes. As illustrated in FIG. 5B, by combining retinol with CBD, the antioxidative and anti-inflammatory effects can be restored through NRF2-independent activation of HMOX1, which plays crucial roles in modulating inflammation, apoptosis, and oxidative stress. 503: Nrf2-independent transcription of HOX1 antioxidative and anti-inflammatory genes. Through the combination of these two agents, the present compositions can combat the intrinsic and extrinsic factors involved in skin aging by increasing skin turnover while protecting the skin through antioxidative and anti-inflammatory mechanisms.

The present compositions can comprise one or more retinoids at a concentration (on a weight basis) of at least 0.01%, or at least 0.1%, or at least 0.5%, or at least 0.75%, or at least 1%, or at least 1.1%, or at least 1.5%, or at least 2%, or at least 3%, or at least 5%, or at least 10%. The present compositions can comprise one or more retinoids at a concentration (on a weight basis) of at most 20%, or at most 15%, or at most 10%, or at most 5%, or at most 4%, or at most 3%, or at most 2.5%, or at most 2%. Any of the foregoing minimum and maximum may be combined to form a desired concentration range, so long as the minimum is smaller than the maximum. When the retinoids is provided in a mixture (such as in a mixture comprising an oil or emulsifier), the weight of the cannabinoid itself (not including the oil or emulsifier or other components in the mixture) is used to calculate its percentage in the composition.

In some embodiments, the present compositions comprise a cannabinoid and a retinoid at a selected weight ratio. For example, the present compositions can have a cannabinoid:retinoid weight ratio that is from 1:2 to 50:1, or from 2:1 to 10:1, or from 3:1 to 7:1, or about 5:1 or 11:2.

The present compositions include one or more aliphatic oligopeptides, such as palmitoyl pentapeptide-4 (also referred to as Pal-Lys-Thr-Thr-Lys-Ser, or Pal-KTTKS). Palmitoyl pentapeptide-4 contains 5 amino acids linked to a 16-carbon aliphatic chain for improving penetration of the peptide-containing molecule through lipid structures of the skin. Palmitoyl pentapeptide-4 is believed to activate certain genes involved in the process of extracellular matrix renewal and cell proliferation. By activating the neosynthesis of extracellular matrix macromolecules, palmitoyl pentapeptide-4 may provide an anti-wrinkle effect. Palmitoyl pentapeptide-4 is commercially available as “Matrixyl” from Sederma SAS (France). Other examples of matrikines include N-Palmitoyl-Gly-His-Lys and N-Palmitoyl-Gly-Gln-Pro-Arg. Various embodiments of the present compositions can include a selected amount of one or more aliphatic oligopeptides, such as at least about 0.5%, 1%, 2%, or 3% by weight, or at most about 15%, 10%, 7% or 5%; any of these minimums and maximums can be combined to form a range for the amount of aliphatic oligopeptides.

The present compositions include one or more acetylated oligopeptides, such as acetyl hexapeptide-3 (also referred to as acetyl hexapeptide-8, Acet-Glu-Glu-Met-Gln-Arg-Arg, or Ac-EEMQRR). Acetyl hexapeptide-8 is commercially available as “Argireline” from Lipotec SAU (Barcelona, Spain). Acetyl hexapeptide-3 is a fragment of Synaptosomal-Associated Protein (SNAP-25), a substrate of botulinum toxin. Acetylated oligopeptides such as acetyl hexapeptide-3 are believed to inhibit reactions that cause muscles to move or contract. Various embodiments of the present compositions can include a selected amount of one or more acetylated oligopeptides, such as at least about 0.5%, 1%, 2%, or 3% by weight, or at most about 15%, 10%, 7% or 5% by weight; any of these minimums and maximums can be combined to form a range for the amount of acetylated oligopeptides.

The present compositions include hyaluronic acid. Hyaluronic acid has been used as a humectant, as it promotes hydration and can hold many times its weight in water. The terms “hyaluronic acid” and “hyaluronate” as used herein are generally equivalent, unless the context indicates otherwise, such as when a specific hyaluronate salt is specified. Furthermore, the terms “hyaluronic acid” and “hyaluronate” generally include hyaluronan, hyaluronate, and glycosaminoglycan. In some embodiments, the present compositions contain a high molecular weight hyaluronic acid and/or a hyaluronic acid which is released over time. In some embodiments, the present compositions contain a selected amount of hyaluronic acid, such as from about 0% to about 5% by weight hyaluronic acid. In some embodiments, the compositions contain at most about 0.1%, or 0.25%, or 0.5%, or 1%, or 1.25%, or 1.5%, or 1.75%, or 2%, or 2.5%, or 3.0% hyaluronic acid. In some circumstances, the foregoing percentages of hyaluronic acid may be on a volume basis.

In some embodiments, the present compositions include one or more humectants (in addition to hyaluronic acid). Humectants can be added to cause increased elasticity, smoothness, and hydration of the skin. Examples of additional humectants include alkyl diols and polyols, alkenyl diols and polyols, alkoxy glycosides, glyceryl triacetate and glycerol polymers, sugars (sucrose, trehalose, fructose, glucose, maltose, etc.), sugar alcohols (erythritol, glycerol, sorbitol, xylitol, maltitol, mannitol, inositol, lactitol, etc.), alkoxy derivatives of sugars and sugar alcohols, alpha hydroxy acids such as lactic acid, and ureas. In some embodiments, the additional humectant is selected from the group consisting of glycerin; propane diols (such as methyl propane diol); propylene glycols (such as dipropylene glycol, triethylene glycol); short-chain polyethylene glycols (<200 mol wt); butylene glycols; pentylene glycols; hexylene glycols (such 1,2,6-hexanetriol); sorbitol, urea compounds; sodium pyrrolidone carboxylic acid; chitosan; and C6-10 alkane diols (such as 1,2-hexanediol, 1,2-octanediol (also called caprylyl glycol), 1,9-nonanediol, 1,2-decanediol, and 1,10-decanediol), and mixtures thereof. In some embodiments, the present compositions contain a selected amount of additional humectant, such as from about 0.05% to about 5% by volume. In some embodiments, the compositions contain at least 0.1% by volume, or 0.25%, or 0.5%, or at most about 2% by volume, or 1%; any of these minimums and maximums can be combined to form a range for the amount of humectant.

In some embodiments, the present compositions can also include any one of, any combination of, or all of the following additional ingredients: a chelating agent, a UV absorption agent, a moisturizing agent, a preservative, a thickening agent, a silicone containing compound, a structuring agent, a vitamin, a pharmaceutical ingredient, or an antioxidant, or any combination of such ingredients or mixtures of such ingredients. In some embodiments, the composition can include at least two, three, four, five, six, seven, eight, nine, ten, or all of these additional ingredients.

In some embodiments, the compositions comprise a solvent in a selected amount. Examples of solvents include water, glycerin, propylene glycol, propanediol, butylene glycol, ethoxydiglycol, isododecane, isohexadecane, octyldodecanol, hexylene glycol, 1,2-hexane diol, and dicaprylyl carbonate, and mixtures thereof. In some embodiments, the solvent is water. The compositions can contain at least 50%, or 60%, or 65%, or 70%, or 75%, or 80% by weight. In some embodiments, the present compositions is about 65 to 70% water. In some circumstances, the foregoing percentages of water may be on a volume basis. In some embodiments, the present compositions are an emulsion, such as an oil-in-water emulsion or a water-in-oil emulsion. In some embodiments, the emulsion is a multiple emulsion, such as water-in-oil-in-water emulsion or an oil-in-water-in-oil emulsion.

In some embodiments, the present compositions comprise a fatty alcohol emollient. Examples of fatty alcohol emollients include capryl alcohol, pelargonic alcohol, capric alcohol, lauryl alcohol, myristyl alcohol, cetyl alcohol, isocetyl alcohol, stearyl alcohol, isostearyl alcohol, cetearyl alcohol, oleyl alcohol, ricinoleyl alcohol, arachidyl alcohol, icocenyl alcohol, behenyl alcohol, and mixtures thereof such as cetearyl alcohol. In some embodiments, the present compositions contain a selected amount of a fatty alcohol emollient, such as from about 0.5% to about 15% by weight. In some embodiments, the compositions contain at least 1% by weight, or 2%, or 3%, or at most about 10% by weight, or 7% w/w, or 5% w/w; any of these minimums and maximums can be combined to form a range for the amount of fatty alcohol emollient. In some circumstances, the foregoing percentages of fatty alcohol emollient may be on a volume basis.

In some embodiments, the present compositions comprise an emulsifier. Emulsifiers can reduce the interfacial tension between phases and improve the formulation and stability of an emulsion. The emulsifiers can be nonionic, cationic, anionic, and zwitterionic emulsifiers. Non-limiting examples include esters of glycerin, fatty acid esters of glycerol such as glyceryl stearate, esters of propylene glycol, fatty acid esters of polyethylene glycol and/or polypropylene glycol such as PEG-100 stearate, salts of fatty acids such as TEA stearate, esters of sorbitol, esters of sorbitan anhydrides, carboxylic acid copolymers, esters of glucose, ethers of glucose, ethoxylated ethers, ethoxylated alcohols, alkyl phosphates, polyoxyethylene fatty ether phosphates, fatty acid amides, acyl lactylates, DEA oleth-3 phosphate, polyethylene glycol 20 sorbitan monolaurate (polysorbate 20), polyethylene glycol 5 soya sterol, steareth-2, steareth-20, steareth-21, ceteareth-20, cetearyl glucoside, cetearyl alcohol, C12-13 pareth-3, PPG-2 methyl glucose ether distearate, PPG-5-ceteth-20, bis-PEG/PPG-20/20 dimethicone, ceteth-10, polysorbate 80, cetyl phosphate, potassium cetyl phosphate, diethanolamine cetyl phosphate, polysorbate 60, arachidyl alcohol, arachidyl glucoside, and mixtures thereof. In some embodiments, the present compositions contain a selected amount of an emulsifier, such as from about 0.5% to about 20% by weight. In some embodiments, the compositions contain at least 1% by weight, or 2%, or 3% of emulsifier, or at most about 15% by weight, or 10%, or 5% of emulsifier; any of these minimums and maximums can be combined to form a range for the amount of emulsifier. In some circumstances, the foregoing percentages of emulsifier may be on a volume basis.

In some embodiments, the present compositions comprise a silicone oil. Examples of silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures thereof. In some embodiments, the present compositions contain a selected amount of a silicone oil, such as from about 0.5% to about 10% by weight. In some embodiments, the compositions contain at least 1% by weight, or 2%, or 3%, or at most about 7% by weight, or 5%; any of these minimums and maximums can be combined to form a range for the amount of silicone oil. In some circumstances, the foregoing percentages of silicone oil may be on a volume basis.

In some embodiments, the present compositions comprise a fragrance, such as a natural oil or a synthetic fragrance. Examples of fragrances include apricot (Prunus armeniaca), chamomile (Anthemis nobilis), coconut (Cocos nucifera), grape (Vitis vinifera), jasmine (Jasminum officinale), lavender (Lavandula angustifolia), lemon (Citrus medica limonum), orange (Citrus aurantium dulcis), peach (Prunus persica), peppermint (Mentha piperita), sage (Salvia officinalis), sandalwood (Santalum album), and combinations thereof. In some embodiments, the present compositions contain a selected amount of a fragrance, such as from about 0.05% to about 5% by weight. In some embodiments, the compositions contain at least 0.1% by weight, or 0.25%, or 0.5%, of fragrance, or at most about 2% by weight, or 1%, of fragrance; any of these minimums and maximums can be combined to form a range for the amount of fragrance.

In some embodiments, the present compositions comprise a preservative. Examples of preservatives include benzalkonium halides such benzalkonium chloride, parabens such as methylparabens and propylparabens, phenoxyethanol, benzyl alcohol, chlorobutanol, phenol, sorbic acid and salts thereof such potassium sorbate, or combinations of two, three, or more of those perservates. In some embodiments, the present compositions contain a selected amount of a preservative, such as from about 0.05% to about 5% by weight. In some embodiments, the compositions contain at least 0.1% by weight, or 0.25%, or 0.5% of a preservative, or at most about 2% by weight, or 1% of a preservative; any of these minimums and maximums can be combined to form a range for the amount of preservative.

In some embodiments, the present compositions are substantially free of one or more cosmetic components which has been used in the past. To some extent, it is surprising that the present compositions are efficacious in reducing signs of aging without including various previously used cosmetic components. In some embodiments, the present compositions are substantially free of, or do not include any detectable amount of, one or more of the following cosmetic components: essential oil such as rose oil; or Aloe vera.

The present compositions can include one or more physiologically acceptable excipients. The term “physiologically acceptable” refers to compounds and materials which are suitable for use in contact with the tissues of human beings and/or other mammals without excessive toxicity, irritation, allergic response, or other problem or complication, commensurate with a reasonable benefit/risk ratio. In some aspects, physiologically acceptable means approved by a regulatory agency of the federal or a state government, or listed in the U.S. Pharmacopeia or other generally recognized pharmacopeia for use in mammals, and more particularly, in humans.

The present compositions can also include other physiologically acceptable excipients which may fall into one or more types. For example, some excipients may be considered both emollients and emulsifiers. Persons skilled in the field of cosmetic formulations will recognize that the identification of an excipient as being within one type does not exclude that same excipient as being within another type. In some embodiments, an excipient may be one type when combined with certain excipients, and another type when used in combination with other excipients.

The present facial cosmetic compositions can be formulated as creams, gels, lotions, or ointments. Creams are generally semi-solid emulsions of oil and water in approximately equal proportions, and can be oil-in-water (O/W) or water-in-oil (W/O) creams. Gels are semi-solid dispersions of liquid or oil particles in a semi-solid medium. Lotions are generally lower in viscosity than creams and gels, and are often oil-in-water emulsions containing an emulsifier such as cetyl alcohol to prevent phase separation. Ointments are generally compositions having a relatively high oil content, such as an oil:water ratio of from 7:1 to 2:1. Serums are

The present compositions can have a desired viscosity which reflects its formulation or its intended use. Depending on the formulation or intended use, the viscosity of the composition can be from about 1 cps to about 1 million cps, or any range based on combining any of the following values: 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps. The foregoing viscosities can be determined on a Brookfield Viscometer using a TC spindle at 2.5 rpm at 25 degrees C.

### Methods of Applying to and Treating Facial Skin

In some embodiments, the present compositions are applied to a subject's facial skin before, during or after a dermatological procedure. Accordingly, the present technology includes methods of improving one or more facial aesthetics of a subject by applying the present composition to the subject's facial skin, and treating the subject's facial skin by one or more other techniques. In some embodiments, the dermatological procedure is selected from the group consisting of microdermabrasion, hydradermabrasion, dermabrasion, dermal planing, massage therapy, administering botulin or other neurotoxins, cosmetic surgery such as brow lifts and blepharoplasty, fillers, micro-needling, peels, exfoliations, suctioning, fluid delivery, acid treatments, extractions, energy-based and other treatments, such as lasers, thermal, radiofrequency, light (e.g., photofacials/IPL), such as light therapy, photodynamic therapy, radio frequency (RF) therapy, or microwave energy therapy. For example, the dermatological procedure can include abrading a surface of facial skin of a subject to remove dead cells and/or cell debris from the skin surface, and this abrading of the facial skin is performed before or during the application of the facial cosmetic composition to the facial skin. The abrading can be performed by techniques such as microdermabrasion and hydradermabrasion. In some embodiments, the facial cosmetic composition is applied to the facial skin by infusion, or by injection or microinjection.

Abrasion as used herein refers to removal or loss of skin tissue and is generally superficial (involving only the epidermis or a sublayer such as the stratum corneum) but in some instances may be deep (involving the deep dermis). Abrasion of the outer layer of the skin or epidermis has been used to smooth or blend scars, blemishes, or other skin conditions. Techniques used for abrasion include dermabrasion and microdermabrasion, which remove portions of the epidermis called the stratum corneum. The body then replaces the lost skin cells, resulting in a new outer layer of skin. Dermabrasion devices may include a wire brush, wheel, sterilized sandpaper, crystals, or other mechanical means.

Microdermabrasion is a technique to temporarily remove top layers of the skin called the stratum corneum, thereby removing skin debris and/or dead cells from the epidermis.

Microdermabrasion uses an exfoliating medium which is applied pneumatically to remove the outermost layer of skin debris and dead cells from the epidermis. Most commonly, microdermabrasion uses two parts: an exfoliating medium comprising crystals or particles, and a pneumatic device applying a vacuum or suction to gently lift up the skin during exfoliation.

Since there may be minor skin sensitivity after microdermabrasion, irritating products like glycolic acids, alpha hydroxy acids, retinoid products, or fragranced creams and lotions are typically avoided right after the procedure. The American Society for Dermatologic Surgery states that glycolic, beta hydroxy acids, retinol, benzyl peroxide and similar medications should be avoided for one to two days following treatment, and applying prescription retinoid products should be avoided for three to seven days after treatment.

Hydradermabrasion combines microdermabrasion with pneumatic application of a composition such as a serum. The serum typically includes an antioxidant. Antioxidants introduced through the procedure hydrate and decrease inflammation in the skin, reversing photo damage, while protecting lipid membranes, collagen fibers, and enzyme systems.

In some embodiments, the present compositions can be used in a dermal infusion treatment. Dermal infusion delivers a composition under pressure to treat dermatological conditions. It has been used to treat rosacea, dehydration, acne, and post-inflammatory hyperpigmentation. In some embodiments, the present composition, in the form of an aqueous suspension or serum, can be filled into a known dermal infusion device and then applied to the skin. The present composition may be applied with or without microdermal abrasion or skin suction.

In some embodiments, the present methods comprise at least partial abrasion of facial skin before, during or after applying the present composition. For instance, a delivery device may comprise one or more abrading members (e.g., posts, spiral, ridges, brushes, sharp edges, roughened surfaces, etc.). When the device is activated (e.g., during the delivery of pulsed air and/or during the maintenance of suction through corresponding vacuum ports, etc.), the user can selectively translate or move the tip of the device relative to the targeted skin surface to at least partially abrade and/or otherwise treat the skin. In other embodiments, the dermal infusion device comprises a non-abrading member for the purpose of driving serums and/or other fluids deeper into the skin. For example, such a non-abrading member can be used following an abrading or preparatory procedure, step or phase.

In some embodiments, the present compositions can be applied using a delivery device, such as the devices described in Ignon et al. US Pat. App. Publication No. 2007/0156124 A1; Boone et al. US Pat. App. Publication No. 2010/0049177 A1; Chang et al. US Pat. App. Publication No. 2015/0088050 A1; Richardson US Pat. App. Publication No. 2018/0140317 A1; Shadduck et al. US Pat. App. Publication No. 2018/0303515 A1; Ignon et al. US Pat. App. Publication No. 2019/0133642 A1; and Straka US Pat. App. Publication No. 2019/0191937 A1.

Before the various examples are described, it is to be understood that the teachings of this disclosure are not limited to the particular examples described, and as such can, of course, vary. In view of this disclosure it is noted that the compositions and methods can be implemented in keeping with the present teachings. Further, various components, materials, steps and parameters are included by way of illustration and example only and not in any limiting sense. In view of this disclosure, the present teachings can be implemented in other applications and components, materials, structures and equipment to implement these applications can be determined, while remaining within the scope of the appended claims.

### EXAMPLES

### Example 1

In this example, an embodiment of the present composition is formulated as a facial cream comprises 1.1% CBD and 0.2% Retinol. The facial cream also comprised the following active agents: 3-5% aliphatic peptide (Matrixyl), 3-5% acetylated peptide (Argireline), and 1-1.5% humectant (Hyaluronic Acid). The facial cream also included the following inactive components: solvent 65-70%; fatty alcohol emollient 3-5%; emulsifier 3-5%; PEG-100 stearate 2-5%; stearic acid 2-5%; silicone oil 2-5%; fragrance 0.5-1%; preservatives 0.5-2%; additional humectant 0.5-1%.

### Example 2

In this example, the cosmetic efficacy, safety and tolerability of the present composition when administered to facial skin was studied. The composition was formulated as a facial cream.

Eligible subjects were those who have received aesthetic treatment or have consulted with the senior investigator (inventor Dr. Julius Few) or the senior investigator's aesthetic providers. Subjects were recruited from the senior investigator's current list of active and former patients. Subjects were healthy females 18 years to 70 years old who presented with facial skin wrinkles and/or facial skin laxity, and met appropriate inclusion criteria and who did not meet exclusion criteria. Subjects who had botulinum toxin or filler within 3 months of study enrollment were excluded. Additionally, subjects who used topical, inhaled, or ingested cannabis and/or hemp-derived products including CBD or tetrahydrocannabinol within 30 days of the study start date or during the study period were excluded. Further key exclusionary characteristics included facial rejuvenation procedures (eg, chemical peels, microneedling, microdermabrasion, etc); pregnancy; advanced or poorly controlled diabetes; current smoker or history of heavy smoking; the use of anti-inflammatory topical products during the study period; or regular continuous use of systemic or topical corticosteroids on the area to be treated).

Table 2 describes characteristics of the ten subjects.

Subject Characteristics (N = 10)

| Characteristic | Value |
|---|---|
| GRS skin type, n (%) | |
| I: pale white or freckled | 1 (10) |
| II: white | 3 (30) |
| III: white to light brown | 3 (30) |
| IV: moderate brown | 1 (10) |
| V: dark brown | 2 (20) |
| VI: very dark brown to black | 0 (0) |

A greater proportion of subjects had either white (30%) or white to light brown (30%) skin compared with other skin types, as determined using the GRS scale. Jain et al. (2017). All 10 subjects reported using the study composition once daily at night before bed for 42 days and product weight was consistent with the high level of compliance reported. No subjects were lost to follow up.

Subjects began treatment immediately after enrollment in the study and continued treatment once daily for 42 days until study completion. The senior investigator conducted safety reviews on a bi-weekly basis, and a nurse practitioner supervised within the study for safety.

Subjects were provided with a tube of a study facial cream described in Example 1 containing 300 mg CBD oil and 0.2% retinol in 30 ml of facial cream. The study cream was labeled “A” by the cosmetic manufacturer. Tubes contained a pump applicator. Subjects were instructed to apply two pumps (roughly a pea sized amount) of the study cream to their entire face nightly after cleansing facial skin with a mild facial cleanser such as Cetaphil or CeraVe. The study cream was to be applied once in the evening before bed for the duration of the study (42 days).

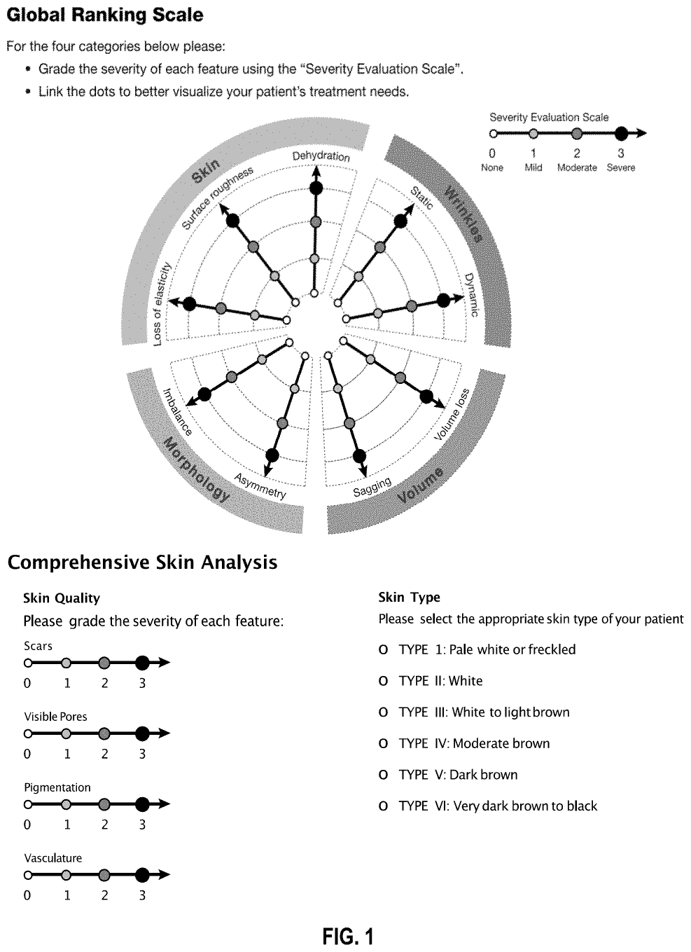

The subjects were to follow the schedule of visits to and assessments with the senior investigator in the table below.

| | |
|---|---|
| Pre-Screen: | Informed Consent-day 1 |
| Subjects will be | Inclusion/Exclusion Criteria-day 1 |
| pre-screened | Medical History-day 1 |
| for the study | Physical Examination-day 1 |
| | Vital Signs, Height, Weight-day 1 |
| | Initial Application Site Reaction Assessment-day 1 |
| Enrollment | Provide and describe daily diary requirements-day 1 |
| | Provide study topical tube-day 1 |
| | Subjects will study and agree to compliance instructions to |
| | participate-day 1 |
| | Subjects will be enrolled in the study the same day of pre- |
| | screen appointment-day 1 |
| Treatment | Nighttime Application to entire face (before bed) after using |
| | a mild facial cleanser (such as Cetaphil and CeraVe) |
| | Subjects will apply one pump of CR topical to the entire |
| | face once daily before bed after facial cleansing |
| | Subjects will record each application in their diary |
| | Subjects will record additional notes (e.g. irritation, side |
| | effects, complaints, aesthetic effects) in diaries on each day |
| | of application (days 1-42) |
| Follow-up | Follow-up appointments will be scheduled-days 1, 21, 42 |
| | Virtual safety assessment-day 3 |
| | Filmed video evaluation of pre-determined phrases-days 1, |
| | 42 |
| | Clinical aesthetic Global Ranking Scale with Senior |
| | investigator (FIG. 1)-days 1, 21, 42 |
| | Subject Satisfaction Questionnaire Completion (see table |
| | below)-days 21, 42 |
| | Review Subject Diary for Compliance-days 21, 42 |
| | Weigh study medication tubes A and B for compliance- |
| | days 1, 21, 42 |
| | Take standardized photographs of treatment areas-days 1, |
| | 21, 42 |
| | Application site reaction assessment-days 1, 21, 42 |
| | Subject’s Self Cosmetic Assessment-days 1, 21, 42 |

The Subject Satisfaction Questionnaire asked the subjects ten questions, eight of which were closed questions with assigned numerical values, and two of which were open-ended questions. The content of the Questionnaire at 3 weeks (21 days) is shown below; the content of the Questionnaire at 6 weeks (42 days) was the same, except for the weeks/days of the questionnaire.

The Questionnaire also provided open space for other comments.

Aesthetic conditions are measured using validated and non-validated scales (eg, FACE-Q, GAIS) using static photos taken from multiple angles. Klassen et al. (2015); Narins et al. (2003). Subject satisfaction questionnaires may also be used to gauge perceptions of efficacy, satisfaction, and overall impressions. However, these scales are limited in their ability to detect specific changes to features such as skin quality. Therefore, the nine-domain Global Ranking Scale (GRS) and four-domain Skin Quality assessment was applied. Jain et al. (2017).

Outcomes were measured at days 1, 21, and 42 using the Global Ranking Scale (GRS) with Comprehensive Skin Analysis by both the subject, blinded evaluator (board certified plastic surgeon) and the senior investigator. Dynamic videos and static imagery were taken before and after treatment and were used to assess efficacy alongside subject satisfaction surveys and diaries. A primary efficacy endpoint for the study was the clinical evaluation of subjects' faces based on comparison of Global Ranking Scale (GRS) scores at each time point, as well as qualitative comparison of standardized before and after photographs on days 1, 21, and 42. During visits on day 1 (baseline), 21, and 42, standardized photographs of the treatment areas were taken and global skin appearance was assessed using the GRS with Comprehensive Skin Analysis by joint evaluation of severity with the lead investigator and the subject. Jain et al. (2017). Dynamic videos and static imagery were taken before and after treatment and were used to assess efficacy alongside subject satisfaction surveys and diaries. The GRS is described in Jain et al. (2017). The components of the GRS are reproduced herein as FIG. 1. The GRS was used to monitor the changes in global skin aesthetic appearance using 13 domains: severity of loss of elasticity, surface roughness, dehydration, static wrinkles/dynamic wrinkles, volume loss, sagging, asymmetry, imbalance, scar presence, visible pores, pigmentation, and vasculature. Each of these 13 GRS domains was graded from 0-3 (0, none; 1, mild; 2, moderate; 3 severe) and mean scored at baseline and at 42 days were calculated for each domain and compared. The senior investigator used a dermascope for grading and factored in qualitative feedback from the subject.

Outcomes were measured by both the subject, a blinded evaluator (board certified plastic surgeon) and the senior investigator. Because the GRS is intended to be used as a collaborative tool by the physician with the patient, it is not conducive to blinded review. Instead, a blinded reviewer was asked to order the static baseline and 42-day images as before and after, and percent agreement with whether the actual photograph was taken at baseline or day 42 was calculated. In addition, questions overlapping with GRS skin quality domains were included in the subject questionnaire to confirm outcomes.

Secondary efficacy endpoints included subject observations from satisfaction questionnaires regarding major cutaneous changes such as smoothness, irritation, pruritus, burning, and erythema. Exploratory endpoints included assessment of 4-dimensional changes among subjects evaluated using simple videos before (baseline) and at study completion (day 42) to assess dynamic and animation-related changes that occurred with treatment. On days 21 and 42, daily subject diaries were reviewed for compliance and tolerability, and subjects completed an 8-question satisfaction survey. Filmed video evaluations occurred on days 1 and 42 for 4-dimensional assessment.

Safety was monitored by the investigator through application site assessment during each follow-up visit (days 1, 21, and 42). Because of the established propensity for retinol to cause irritation, an additional, virtual safety assessment of the application site occurred on day 3. At each follow-up visit, study medication tubes were weighed for compliance. This study adhered to the Good Clinical Practice and standards set forth in the World Medical Association's Declaration of Helsinki. Consent for treatment and all included photographs and video were obtained.

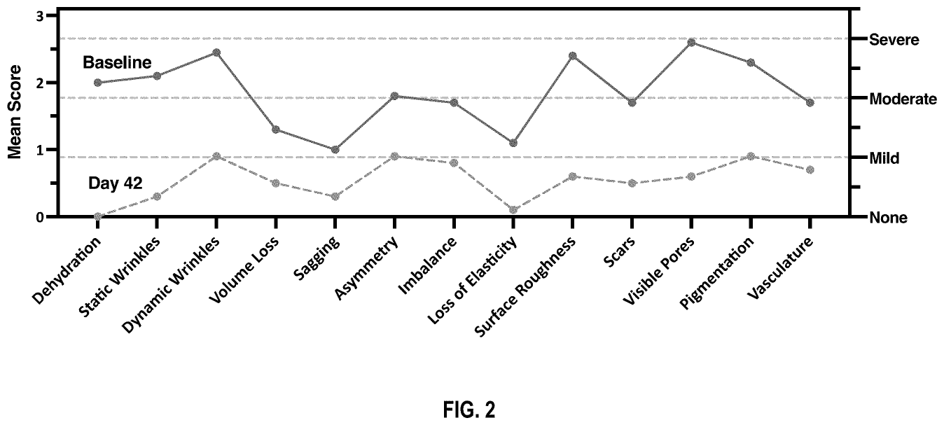

Results: The investigator's evaluations of the subject's outcomes in the study are summarized in FIGS. 2 to 4. Each subject served as their own control, and paired differences for each of the 13 AGRS domains between day 1 and day 42 were determined. Each of the scale's 13 domains, including loss of elasticity, surface roughness, dehydration, static and dynamic wrinkles, volume loss, sagging, asymmetry, imbalance, scar presence, visible pores, pigmentation, and vasculature were graded from 0-3 (0, none; 1, mild; 2, moderate; 3, severe), and mean scores at baseline and at 42 days were calculated for each domain and compared. Jain et al. (2017). Paired mean difference with each subject used as their own control, and lower and upper 95% confidence intervals for each domain was calculated. Due to the small number of subjects and the use of a 4-point scale across numerous domains, paired t-tests with p-values are less informative (p-values calculated using a paired two-tailed t-test were significant for all measures assessed), and are not presented here.

The differences in GRS parameters from the 10 subjects were averaged to provide an average difference for each of the GRS parameters. The blinded evaluator also completed the GRS based on before-and-after photographs, and the magnitudes of the paired difference were less than those from the senior investigator. It was subsequently recognized that the blinded evaluator had not received adequate training in the use of the GRS.

FIG. 2 is a line graph that illustrates the mean differences for each of the 13 GRS domains. The top line is drawn between the data points for day 1 and the bottom line is drawn between data points for day 42. At 42 days, GRS with Comprehensive Skin Analysis score improvement was observed from baseline to day 42 across all 13 domains, including domains for which improvement was unexpected (for example, sagging). While improvement was consistent, some areas improved more than others, generally those most consistent with a topical treatment (FIG. 2). Among subjects, the highest mean scores at baseline were visible pores (2.6), dynamic wrinkles (2.5), surface roughness (2.4), pigmentation (2.3), and static wrinkles (2; Higher mean scores correlate to worse skin quality [0, none; 1, mild; 2, moderate; 3, severe]).

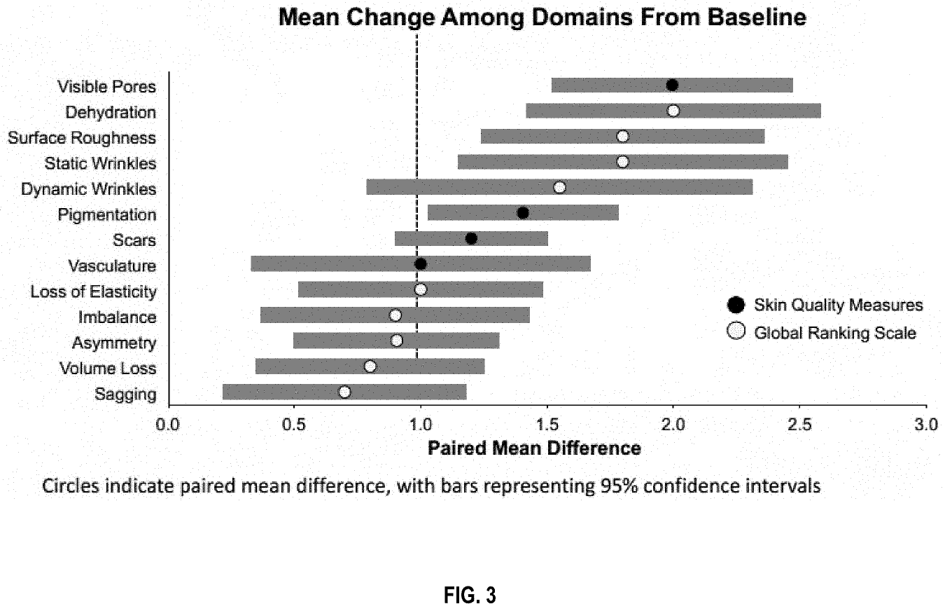

FIG. 3 shows mean change among domains from baseline, with confidence intervals around the paired differences. All of the GRS domains decreased from day 1 to day 42. When assessing the relative degree of improvement for each domain, those with 1-point improvement from day 0 to day 42 were found to be consistent with topical treatments (FIG. 3, black dotted line). While statistically significant changes were observed for each domain including both the GRS and skin quality measures, the areas of greatest change were visible pores (2.0-point change; 95% CI, 1.5-2.5), dehydration (2.0-point change; 95% CI, 1.4-2.6), surface roughness (1.8-point change; 95% CI, 1.2-2.4) static wrinkles (1.8-point change; 95% CI, 1.1-2.5), and dynamic wrinkles (1.6-point change; 95% CI, 0.8-2.3). Notably, while the greatest possible score change is 3, not all subjects began with “severe” skin quality, making a 3-point change impossible. However, a two-point change represents a change from “severe” to “mild” or “moderate” to “none,” both of which represent very impactful improvements. Each subject who reported severe static wrinkles, dynamic wrinkles, surface roughness, scars, visible pores, or vasculature at baseline reported at least “mild” severity for these domains at study completion. Moreover, all subjects with “severe” dehydration reported “none” at the end of the study. The blinded reviewer correctly ordered before and after images for 80% of the subjects.

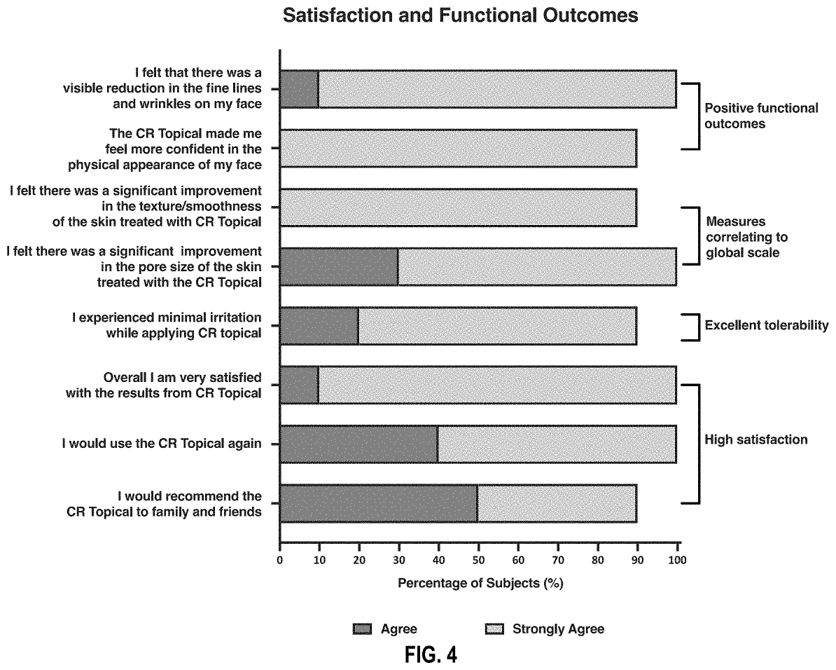

FIG. 4 illustrates satisfaction and functional outcomes assessed using subject questionnaires completed on days 21 and 42. At day 42, 90% of subjects either agreed (50%) or strongly agreed (40%) that they experienced visible reduction of facial fine lines and wrinkles. Overall, 100% of subjects felt that the study facial cream made them more confident in the physical appearance of their face. In agreement with GRS outcomes, subjects strongly agreed that the use of CR Topical significantly improved the texture/smoothness (90%) and pore size (70%) of the skin, outcomes consistent with those measured with the GRS. Subjects either agreed or strongly agreed (30% vs 70%, respectively) with the statement that they experienced minimal irritation for the duration of the study, demonstrating the excellent tolerability of CR Topical. Furthermore, 90% of subjects strongly agreed that they were very satisfied with the product, with 90% expressing both their willingness to use CR Topical again and recommend the cream to family and friends.

Representative patient images, along with 4-dimensional videos before and after treatment, illustrate improvement in both static and dynamic lines, respectively. In the four-dimensional assessment using videos, improvement in skin quality and texture/shadowing is apparent as the subject's facial expressions change, as are subjective changes in subject confidence and openness in expressions. The photographs and video evaluations of the subjects' face are not included herein.

Discussion. This study demonstrated that the present composition comprising a cannabinoid such as CBD, a retinoid such as retinol, peptides, and antioxidants, increases global skin quality and leads to positive subject functional outcomes. While retinol is correlated to a decrease in the depth of fine lines and wrinkles, it is known to have irritating effects on the skin that can lead to erythema, pruritus, peeling, and redness with long-term use. Kim et al. (2003). Here, these negative effects are counteracted by cannabidiol, improving the tolerability of the product. Although research about CBD oil as a topical agent is still emerging, supplementary use has been shown to decrease inflammation and improve therapeutic outcomes for severe inflammatory skin diseases, supporting this potential role. Sholler et al. (2020); Palmieri et al. (2019); Oláh et al. (2014).

The improvements observed for the combination of retinol and CBD are better than for either product alone. This suggests at least an additive, or potentially synergistic benefit. Larger controlled studies have the potential to provide greater understanding of the relative effects of a cannabinoid and a retinoid, and to define any synergistic activity for the two ingredients.

Without being bound by theory, it is presently believed that the combination of a cannabinoid and a retinoid has a synergistic effect in improving facial skin aesthetics. When considering the potential for synergistic activity, the mechanism of action for broth retinol and CBD is informative. Through the combined effects of promoting cell proliferation and reducing oxidative stress, these agents may work synergistically to combat the extrinsic and extrinsic causes of aging. While the mechanism of action of CBD oil in the skin has yet to be fully elucidated, its antioxidant and anti-inflammatory activities are well recognized, and can be partially attributed to its activation of Nrf2-target activation genes, including HMOXJ, which plays crucial roles in modulating inflammation, apoptosis, and oxidative stress. Casares et al. (2020). Importantly, CBD-induced activation of HMOX1 expression occurs through a Nrf2-independent mechanism. Casares et al. (2020). Because retinol suppresses expression of Nrf2-target antioxidative genes, the addition of CBD oil can potentially restore antioxidative effects inhibited by retinol while allowing for retention of skin strengthening properties. Zasada et al. (2019); Wang et al. (2013); Oláh et al. (2014). This activity of CBD is in addition to independent mechanisms, including modulation of the human endocannabinoid system (ECS), which is linked to anti-aging effects through its role in maintaining skin homeostasis and barrier function, antioxidant and anti-inflammatory activity, as well optimal sebum production. Oláh et al. (2014); Casares et al. (2020); Baswan et al. (2020). For Retinol, independent effects are manifested through promoting keratinocyte proliferation, strengthening of the epidermis, and increasing collagen (through protection of existing collagen and stimulation of neocollagenesis) in the skin. Zasada et al. (2019); Hubbard et al. (2014). Importantly, the combination of CBD and retinol appears to reduce skin irritation often observed with retinol alone. Notably, 100% of subjects who reported “severe” dehydration at baseline reported “none” at the conclusion of the study, indicating that CBD oil may combat the irritation characteristic of retinol topicals, providing considerable benefit to subject tolerability and indirect improvement in efficacy due to increasing the likelihood of adherence.

While improvement across all 13 domains for all subjects suggests a global effect of the present composition, improvements in areas such as sagging or asymmetry may signal some variability in day-to-day perceptions, some level of bias that stems from the investigator and the subject completing the assessment together, or incomplete ability of the subject to differentiate entirely between the contributions of individual domains to overall global improvement. In future studies, placebo control and blinding both the subject and evaluating investigator will shed light on this topic. Additionally, when interpreting results, it is important to consider that the rating scale ranged from 0 to 3. Therefore, seemingly modest changes (eg, “severe” at 3 to “mild” at 1) do indeed reflect a significant improvement. A broader scale range (eg, a 5-point scale) may be considered for future studies. Because of the granular nature of the GRS scale, it is possible that future studies could also evaluate combination treatment with other products or modalities versus monotherapy, further characterizing the effects of distinct interventions on global outcome when used together.

Historical measurements for skin treatment efficacy have relied on static imagery, this methodology cannot capture the movement of glide planes and facial expressions, and it is how our faces move during communication that most informs perceptions of beauty. Capturing movements of the face with short videos more accurately reflects what is observed in day-to-day life as opposed to static imagery, and provides a real-world context for evaluating the most relevant effects of treatment. This novel and dynamic 4-dimensional assessment may prevent overcorrection or unnatural looking results and can be used in conjunction with new or existing other scales and opens the door for incorporation of new measures rating naturalness of outcomes, projection or correction across multiple facial expressions, apparent confidence, openness, or other characteristics that are not traditionally thought of as study outcomes. Moreover, subjects can use the videos to better evaluate their overall satisfaction and experience by observing their facial movements before and after treatment. Like the GRS, 4-dimensional assessment can be used as a tool for patient education, treatment planning, and encouraging pursuit of natural-looking results.

The exploratory assessment with 4-dimensional dynamic imagery was successful in capturing skin quality improvement across multiple domains among subjects using the present composition, including both static and dynamic wrinkles. The GRS was used in conjunction with static and 4-dimensional dynamic imagery to obtain a comprehensive efficacy profile.

Shortcomings of this study include the small sample size and short duration of follow-up time, nonetheless, the results are significant and indicative of the composition's efficacy. A larger cohort and increased length of the study could lead to more robust results. Future studies could compare the efficacy of the present composition with retinol alone.

### Example 3

In this example, an embodiment of the present composition is formulated as a serum for use with a hydrafacial device. The serum comprises 1.1% CBD and 0.2% Retinol. The facial cream also comprises the following active agents: 3-5% aliphatic peptide (Matrixyl), 3-5% acetylated peptide (Argireline), and 2-2.5% humectant (Hyaluronic Acid). The facial serum also includes inactive components, such as water 80-85% and 0.5-1% preservative.

### Example 4

In this example, the serum of Example 3 is applied to facial skin of a subject using a hydradermabrasion device. The subject experiences less irritation and redness than when conventional serums are applied.

As used in the specification and appended claims, and in addition to their ordinary meanings, the terms “substantial” or “substantially” mean to within acceptable limits or degree to one having ordinary skill in the art. For example, “substantially free of” a material means that one skilled in the art considers the remaining amount of material to be acceptable.

As used in the specification and the appended claims and in addition to its ordinary meaning, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same.

In the present disclosure, when percentages are used to identify the amount of a component, the percentages are based on the weight of the component over the total weight of the composition (unless the context indicates another basis of calculating the percentage). When a component is provided in a mixture (such as in a mixture comprising a liquid medium), the weight of the component itself (not including the liquid medium or other components in the mixture) is used to calculate its percentage in the composition.

As used in the specification and appended claims, the terms “a,” “an,” and “the” include both singular and plural referents, unless the context clearly dictates otherwise. Thus, for example, “a retinoid” includes one retinoid and multiple retinoids.

In the present disclosure, numeric ranges are inclusive of the numbers defining the range. It should be recognized that chemical structures and formula may be elongated or enlarged for illustrative purposes.

As disclosed herein, a number of ranges of values are provided. Each smaller range between any stated value or intervening value in a stated range and any other stated or intervening value in that stated range is encompassed within the invention. The upper and lower limits of these smaller ranges may independently be included or excluded in the range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

All patents and publications referred to herein are expressly incorporated by reference.

### EXEMPLARY EMBODIMENTS

Exemplary embodiments provided in accordance with the presently disclosed subject matter include, but are not limited to, the following:

Embodiment 1. A facial cosmetic composition comprising: (a) a cannabinoid; and (b) retinoid.

Embodiment 2. The composition of embodiment 1, wherein the cannabinoid is selected from the group consisting of cannabidiol (CBD), precursors and derivatives of cannabidiol, and mixtures thereof.

Embodiment 3. The composition of embodiment 1, wherein the cannabinoid is selected from the group consisting of cannabidiol (CBD), Cannabidiolic Acid (CBDA), and mixtures thereof.

Embodiment 4. The composition of embodiment 1, wherein the retinoid is selected from the group consisting of retinol, retinal, retinyl esters, and mixtures thereof.

Embodiment 5. The composition of any of the foregoing embodiments, further comprising (c) an aliphatic oligopeptide.

Embodiment 6. The composition of embodiment 5, wherein the aliphatic oligopeptide is a palmitoyl oligopeptide.

Embodiment 7. The composition of any of the foregoing embodiments, further comprising (d) an acetylated oligopeptide.

Embodiment 8. The composition of embodiment 7, wherein the acetylated oligopeptide is an acetyl hexapeptide.

Embodiment 9. The composition of any of the foregoing embodiments, further comprising (e) hyaluronic acid.

Embodiment 10. The composition of any of the foregoing embodiments, wherein the composition is substantially free of psychoactive cannabinoids.

Embodiment 11. A method of improving one or more facial aesthetics of a subject, comprising: applying to facial skin of a subject the facial cosmetic composition of any of the foregoing embodiments, wherein the composition is applied at least once per day for at least 21 days.

Embodiment 12. The method of embodiment 11, wherein the composition is applied at least once per day for at least 42 days.

Embodiment 13. The method of embodiment 11, wherein the composition is a cream applied to the facial skin without removal.

Embodiment 14. A method of improving one or more facial aesthetics of a subject, the method comprising: applying a facial cosmetic composition to facial skin of a subject, wherein the composition comprises: (a) a cannabinoid; and (b) a retinoid; and treating the facial skin of the subject by a dermatological procedure before, during or after applying the facial cosmetic composition.

Embodiment 15. The method of embodiment 14, wherein the composition further comprises one or more of (c) an aliphatic oligopeptide; (d) an acetylated oligopeptide; and (e) hyaluronic acid.

Embodiment 16. The method of embodiment 14 or 15, wherein the facial skin treatment is selected from the group consisting of microdermabrasion, hydradermabrasion, dermabrasion, dermal planing, and combinations thereof.

Embodiment 17. The method of embodiment 14 or 15, wherein the facial skin treatment comprises abrading a surface of facial skin of a subject to remove dead cells and/or cell debris from the skin surface; wherein said abrading of the facial skin is performed before or during said applying the facial cosmetic composition to the facial skin.

Embodiment 18. The method of embodiment 17, wherein said abrading is by microdermabrasion.

Embodiment 19. The method of embodiment 17, wherein said abrading is by hydradermabrasion.

Embodiment 20. The method of embodiment 14 or 15, wherein the facial cosmetic composition is applied to the facial skin by infusion.

Embodiment 21. The method of embodiment 14 or 15, wherein the facial cosmetic composition is applied to the facial skin by injection or microinjection.

It will be understood that various aspects or details of the invention may be changed without departing from the scope of the invention. Furthermore, the foregoing description is for the purpose of illustration only, and not for the purpose of limitation—the invention being defined by the claims.

### REFERENCES

Zhang S, Duan E. Fighting against skin aging: the way from bench to bedside. Cell Transplant. 2018; 27(5): 729-738.
Dimri G P, Lee X, Basile G, et al. A biomarker that identifies senescent human cells in culture and in aging skin in vivo. Proc Natl Acad Sci USA. 1995; 92(20):9363-9367.
Kohl E, Steinbauer J, Landthaler M, Szeimies R M. Skin ageing. J Eur Acad Dermatol Venereol. 2011; 25(8):873-884.
Friedman O. Changes associated with the aging face. Facial Plast Surg Clin North Am. 2005; 13(3):371-380.
Rinnerthaler M, Bischof J, Streubel M K, et al. Oxidative stress in aging human skin. Biomolecules. 2015; 5(2):545-589.
Tsuchida K, Kobayashi M. Oxidative stress in human facial skin observed by ultraweak photon emission imaging and its correlation with biophysical properties of skin. Sci Rep. 2020; 10(1):9626.
Tóth K F, et al., Cannabinoid Signaling in the Skin: Therapeutic Potential of the “C(ut)annabinoid” System. Molecules. 2019; 24(5):918.
Atalay S, et al., Antioxidative and Anti-Inflammatory Properties of Cannabidiol. Antioxidants (Basel). 2019; 9(1):E21.
Petrosino S, et al., Anti-inflammatory Properties of Cannabidiol, a Nonpsychotropic Cannabinoid, in Experimental Allergic Contact Dermatitis. J Pharmacol Exp Ther. 2018; 365(3):652-663.
Saha S, et al., An Overview of Nrf2 Signaling Pathway and Its Role in Inflammation. Molecules. 2020; 25(22):E5474.
Zasada M, et al., Retinoids: active molecules influencing skin structure formation in cosmetic and dermatological treatments. Adv Dermatol Allergol. 2019; 36(4):392-397.
Hubbard B A, et al., Reversal of skin aging with topical retinoids. Plast Reconstr Surg. 2014; 133(4):481e-490e.
Wang X J, et al., Identification of retinoic acid as an inhibitor of transcription factor Nrf2 through activation of retinoic acid receptor alpha. Proc Natl Acad Sci USA. 2007; 104(49): 19589-19594.
Wang H, et al., RXRα Inhibits the NRF2-ARE Signaling Pathway through a Direct Interaction with the Neh7 Domain of NRF2. Cancer Res. 2013; 73(10):3097-3108.
Bouriez D, et al., Efficiency of All-Trans Retinoic Acid on Gastric Cancer: A Narrative Literature Review. Int J Mol Sci. 2018; 19(11):E3388.
An J H, et al., Anti-Wrinkle Efficacy of Cross-Linked Hyaluronic Acid-Based Microneedle Patch with Acetyl Hexapeptide-8 and Epidermal Growth Factor on Korean Skin. Ann Dermatol. 2019; 31(3):263-271.
Talalaj U, et al., The Effects of a Novel Series of KTTKS Analogues on Cytotoxicity and Proteolytic Activity. Molecules. 2019; 24(20):3698.
Mukherjee S, Date A, Patravale V, et al. Retinoids in the treatment of skin aging: an overview of clinical efficacy and safety. Clin Intery Aging. 2006; 1(4):327-348.
Zasada M, Budzisz E. Retinoids: active molecules influencing skin structure formation in cosmetic and dermatological treatments. Postepy Dermatol Alergol. 2019; 36(4):392-397.
Hubbard B A, Unger J G, Rohrich R J. Reversal of skin aging with topical retinoids. Plast Reconstr Surg. 2014; 133(4):481e-490e.
Wang H, Liu K, Geng M, et al. RXRα inhibits the NRF2-ARE signaling pathway through a direct interaction with the Neh7 domain of NRF2. Cancer Res. 2013; 73(10):3097-3108.
Klassen A F, Cano S J, Schwitzer J A, Scott A M, Pusic A L. FACE-Q scales for health-related quality of life, early life impact, satisfaction with outcomes, and decision to have treatment: development and validation. Plast Reconstr Surg. 2015; 135(2):375-386.
Narins R S, Brandt F, Leyden J, Lorenc Z P, Rubin M, Smith S. A randomized, double-blind, multicenter comparison of the efficacy and tolerability of Restylane versus Zyplast for the correction of nasolabial folds. Dermatol Surg. 2003; 29(6):588-595.
Jain R, Huang P, Ferraz R M. A new tool to improve delivery of patient-engaged care and satisfaction in facial treatments: the Aesthetic Global Ranking Scale. J Cosmet Dermatol. 2017; 16(1):132-143.
Cartier H, Hedén P, Delmar H, et al. Repeated full-face aesthetic combination treatment with abobotulinumtoxinA, hyaluronic acid filler, and skin-boosting hyaluronic acid after monotherapy with abobotulinumtoxinA or hyaluronic acid filler. Dermatol Surg. 2020; 46(4):475-482.
Kim B H, Lee Y S, Kang K S. The mechanism of retinol-induced irritation and its application to anti-irritant development. Toxicol Lett. 2003; 146(1):65-73.
Sholler D J, Schoene L, Spindle T R. Therapeutic efficacy of cannabidiol (CBD): a review of the evidence from clinical trials and human laboratory studies. Curr Addict Rep. 2020; 7(3):405-412.
Palmieri B, Laurino C, Vadalà M. A therapeutic effect of cbd-enriched ointment in inflammatory skin diseases and cutaneous scars. Clin Ter. 2019; 170(2):e93-e99.
Oláh A, Tóth B I, Borbiró I, et al. Cannabidiol exerts sebostatic and antiinflammatory effects on human sebocytes. J Clin Invest. 2014; 124(9):3713-3724.
Casares L, Garcia V, Garrido-Rodriguez M, et al. Cannabidiol induces antioxidant pathways in keratinocytes by targeting BACH1. Redox Biol. 2020; 28:101321.
Baswan S M, Klosner A E, Glynn K, et al. Therapeutic potential of cannabidiol (CBD) for skin health and disorders. Clin Cosmet Investig Dermatol. 2020; 13:927-942.

What is claimed is:

1. A facial cosmetic composition comprising:
(a) a cannabinoid; and
(b) a retinoid.

2. The composition of claim 1, wherein the cannabinoid is selected from the group consisting of cannabidiol (CBD), precursors and derivatives of cannabidiol, and mixtures thereof.

3. The composition of claim 1, wherein the cannabinoid is selected from the group consisting of cannabidiol (CBD), Cannabidiolic Acid (CBDA), and mixtures thereof.

4. The composition of claim 1, wherein the retinoid is selected from the group consisting of retinol, retinal, retinyl esters, and mixtures thereof.

5. The composition of claim 1, further comprising (c) an aliphatic oligopeptide.

6. The composition of claim 5, wherein the aliphatic oligopeptide is a palmitoyl oligopeptide.

7. The composition of claim 1, further comprising (d) an acetylated oligopeptide.

8. The composition of claim 7, wherein the acetylated oligopeptide is an acetyl hexapeptide.

9. The composition of claim 1, further comprising (e) hyaluronic acid.

10. The composition of claim 1, wherein the composition is substantially free of psychoactive cannabinoids.

11. A method of improving one or more facial aesthetics of a subject, comprising:
applying to facial skin of a subject the facial cosmetic composition of claim 1, wherein the composition is applied at least once per day for at least 21 days.

12. The method of claim 11, wherein the composition is applied at least once per day for at least 42 days.

13. The method of claim 11, wherein the composition is a cream applied to the facial skin without removal.

14. A method of improving one or more facial aesthetics of a subject, the method comprising:
applying a facial cosmetic composition to facial skin of a subject, wherein the composition comprises:
(a) a cannabinoid; and
(b) a retinoid; and
treating the facial skin of the subject by a dermatological procedure before, during or after applying the facial cosmetic composition.

15. The method of claim 14, wherein the composition further comprises one or more of (c) an aliphatic oligopeptide; (d) an acetylated oligopeptide; and (e) hyaluronic acid.

16. The method of claim 14, wherein the facial skin treatment is selected from the group consisting of microdermabrasion, hydradermabrasion, dermabrasion, dermal planing, and combinations thereof.

17. The method of claim 14, wherein the facial skin treatment comprises abrading a surface of facial skin of a subject to remove dead cells and/or cell debris from the skin surface;
wherein said abrading of the facial skin is performed before or during said applying the facial cosmetic composition to the facial skin.

18. The method of claim 17, wherein said abrading is by microdermabrasion or by hydradermabrasion.

19. The method of claim 14, wherein the facial cosmetic composition is applied to the facial skin by infusion.

20. The method of claim 14, wherein the facial cosmetic composition is applied to the facial skin by injection or microinjection.
